(19) United States
(12) Patent Application Publication
(10) Pub. No.: US 2014/0190479 A1
(43) Pub. Date: Jul. 10, 2014

(54) SPEAKING VALVE WITH SATURATION INDICATION

(71) Applicant: NELLCOR PURITAN BENNETT LLC, Boulder, CO (US)

(72) Inventors: Kamlesh Sethiya, C. Westmeath, Athlone (IR); Emmet Bolger, C. Westmeath, Athlone (IR)

(73) Assignee: NELLCOR PURITAN BENNETT LLC, Boulder, CO (US)

(21) Appl. No.: 13/738,834

(22) Filed: Jan. 10, 2013

(51) Int. Cl. A61M 16/04; A61M 16/20; A61M 16/06; A61M 16/10; A61M 16/14; A61M 16/00; A61M 16/08

(52) U.S. Cl. CPC A61M 16/0468; A61M 16/0051; A61M 16/20; A61M 16/0434; A61M 16/08; A61M 16/105; A61M 16/14; A61M 16/0666; A61M 16/0057; A61M 16/0472
USPC 128/202.22

(57) ABSTRACT

A speaking valve includes a hollow body configured to be secured to a connector of a tracheostomy system disposed in a patient. The speaking valve further includes a cap that houses a valve member, which enables the speaking valve to act as a one-way check valve. The valve member, hollow body, or other components of the valve may include a material that undergoes a chemical changed when exposed to moisture, i.e., a hydrosensitive material. Particularly, the hydrosensitive material may exhibit a color change when exposed to moisture. As such, the hydrosensitive material may be useful in providing a visual indication of the saturation and/or moisture level of the speaking valve.

### BACKGROUND

The present disclosure relates generally to medical devices and, more particularly, to airway devices, such as breathing and speaking valves for tracheostomy tubes.

This section is intended to introduce the reader to aspects of the art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the course of treating a patient, a tube or other medical device may be used to control the flow of air, food, fluids, or other substances into the patient. For example, tracheal tubes may be used to control the flow of air or other gases through a patient's trachea and into the lungs, particularly during patient ventilation. Such tracheal tubes may include tracheostomy tubes, which provide direct access to the patient's lungs via incisions in the neck and trachea of the patient. In some instances, it is desirable to provide the patient with the ability to breathe and/or speak of their own accord while the tracheostomy tube is still disposed within the trachea. In this way, patients that are tracheally intubated can begin to build the strength to breathe and speak independently in a clean and controlled environment.

To provide the patient the ability to breathe and speak, a one-way valve may be disposed over an end of the tracheostomy tube that is external to the patient. Once in place, the one-way valve generally permits airflow to travel in only one direction within the tracheostomy tube. When the patient inhales, the check valve opens to allow air into the lungs. However, when the patient exhales, the check valve closes to enable the exhalation air to exit via the mouth and/or nose to facilitate speaking and breathing. The patient may use the valve for an extended period of time, and during that time the valve is subjected to moisture in the form of contaminants (e.g., mucus, secretions, saliva, etc.) from the tracheostomy site and humidity from the inhalation and exhalation air. Moisture can accumulate within the valve, which may lead to reduced valve performance, changed patient breathing cycles, and unsanitary conditions within the valve.

There is a need, therefore, for improved speaking valves, particularly with respect to determining the moisture and/or saturation level within the valve. Accordingly, it may be desirable to have the ability to visually monitor the saturation level in the valve to determine when the valve needs to be cleaned and/or changed.

### BRIEF DESCRIPTION

This disclosure provides a novel one-way check valve, for enabling breathing and/or speaking in a patient having a tracheostomy tube, designed to respond to such needs. Particularly, the valve may include a means of visually indicating the moisture level within the valve.

The one-way check valve may be fitted over a connector of a tracheostomy tube, disposed in a patient, to provide a unidirectional path for airflow into the patient's lungs. During inhalation, the air flows through a cannula into the lungs. During exhalation, the air may not exit the patient via the cannula, but may be directed through the trachea to exit through the nose and/or mouth. In this way, the valve may force the exhalation air through the vocal folds, enabling the patient to speak while intubated. To function properly, the valve must remain clean and unobstructed; otherwise, the valve may impede patient breathing or create an unsanitary environment within the breathing circuit. Typical causes of valve occlusion and contamination include moisture from inhalation and exhalation air, mucus, tracheostomy site secretions, and saliva. Thus, speaking valves must be inspected regularly by patients or caretakers to ensure that moisture does not build up within the valve. This adds to the workflow of the caretakers and results in excessive handling of the tracheostomy system and site, which may lead to patient discomfort and infection of the site.

Accordingly, the disclosed embodiments provide a passive system for monitoring the saturation and moisture level within a speaking valve by incorporating a material susceptible to moisture, i.e., a hydrosensitive material. Specifically, the speaking valve having the hydrosensitive material may be inspected visually to determine the moisture level within the valve, eliminating the need to physically handle the valve during inspection. The hydrosensitive material may undergo a color change when it reaches a specified saturation point. Various materials may be suitable for use as the hydrosensitive material, such as temperature responsive polymers, pH sensitive polymers, halochromic materials, chromogenic materials, hydrogels, thermochromic materials, lichens based materials, other materials that may exhibit a color change due to exposure to moisture, or a combination thereof.

Such a color change may be gradual to indicate an increasing amount of moisture build-up within the valve, with the completion of the color change indicating complete saturation of the hydrosensitive material. In this way, the color change of the hydrosensitive material may provide a visual indication to the patient and/or a caretaker that the speaking valve needs to be cleaned or changed. Specifically, the valve may be cleaned or changed prior to the patient experiencing negative effects due to moisture build-up and without physically handling or removing the valve from the patient.

In certain embodiments, a body component of the speaking valve may be constructed from the hydrosensitive material. Once the color change of the body reaches a defined point (e.g., complete color change, partial color change, etc.), the speaking valve may be discarded and replaced with a new speaking valve. In other embodiments, the speaking valve may include at least one internal valve member constructed of the hydrosensitive material. The valve member may be removed and cleaned or replaced as it becomes saturated, as indicated by the color change. As only the internal valve member is being removed, the speaking valve can remain connected to the patient and the body of the valve may be reusable, resulting in reduced cost and waste.

### BRIEF DESCRIPTION OF THE DRAWINGS

Advantages of the disclosure may become apparent upon reading the following detailed description and upon reference to the drawings in which:

FIG. 1 illustrates a patient having a tracheostomy system with a speaking valve that includes a hydrosensitive material for monitoring the saturation of valve components according to embodiments of the present techniques;

FIG. 2 is a perspective view of a tracheostomy system with a speaking valve having hydrosensitive components that may be disposed in the patient of FIG. 1;

FIG. 3 is a perspective view of a speaking valve that may contain components constructed from a hydrosensitive material;

FIG. 4 is an exploded view of an embodiment of a valve member constructed from membranes of hydrosensitive materials that may provide a visual saturation indicator in conjunction with the valve of FIG. 3;

FIG. 5 is a side view of a membrane of FIG. 4 constructed from a hydrosensitive material that may provide one-way deflection for the valve of FIG. 3; and

FIG. 6 is a flow diagram depicting an embodiment of a method of use of a valve with hydrosensitive components.

### DETAILED DESCRIPTION OF SPECIFIC EMBODIMENTS

One or more specific embodiments of the present disclosure will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

In certain embodiments, the disclosed speaking valves, systems, and methods may be used in conjunction with any appropriate medical device, including a tracheostomy tube, a tracheal tube, an endotracheal tube, a double-lumen tracheal tube, a circuit, an airway accessory, a connector, an adapter, a filter, a nebulizer, a nasal cannula, or a supraglottal mask/tube. The present techniques may also be used in conjunction with any of the listed types of tracheal tubes having an inflatable cuff.

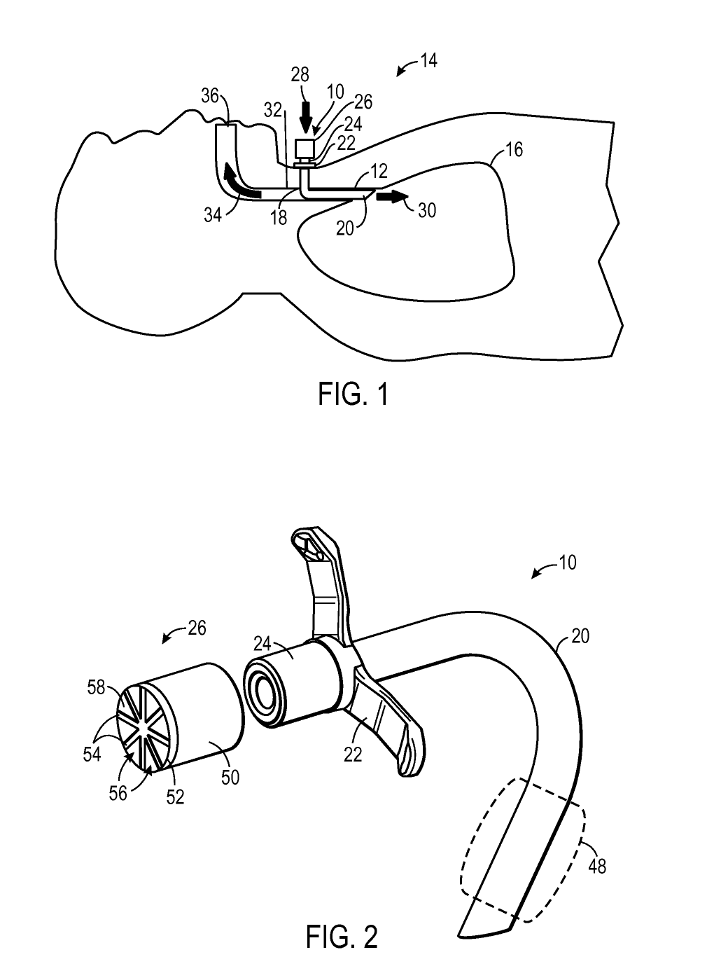

Turning now to the drawings, FIG. 1 shows a tracheostomy system 10 that has been inserted into the trachea 12 of a patient 14. The system 10 provides controlled access to the lungs 16 of the patient 14 via a tracheostomy site 18 on the anterior portion of the neck. The system 10 includes a cannula 20 that provides a fluid pathway to the lungs 16. A flange 22 is disposed near the proximal end of the cannula 20 and rests on the anterior portion of the neck to provide stability to the system 10. At the proximal tip of the cannula 20, a connector 24 provides a connection point for attaching additional airway accessories to the system 10. Such an accessory may be a speaking valve 26, which enables the patient 14 to speak and breathe independently while the system 10 is disposed in the patient 14. As detailed below, the speaking valve 26 may include a material that undergoes a chemical change when exposed to moisture, resulting in a change of color.

To enable speaking, the valve 26 acts as a one-way check valve and allows only inhalation air, indicated by arrow 28, to travel through the cannula 20. The inhalation air exits the distal end of the cannula 20 and enters the lungs 16, as indicated by arrow 30. As exhalation begins, the valve 26 blocks the air from exiting the patient 14 via the cannula 20, thereby forcing the air to pass the larynx 32, as indicated by arrow 34. The larynx 32 houses vocal folds, which vibrate as the air (following arrow 34) flows past. Vibration of the vocal folds facilitates phonation, i.e., speaking When speaking, the exhalation air exits the patient 14 via the mouth 36.

A detailed perspective view of the tracheostomy system 10 and the speaking valve 26 is provided in FIG. 2. It should be understood that the embodiments discussed herein may be implemented with any suitable airway device, which may include an inflatable cuff 48. However, because the speaking valve 26 prevents air from exiting the patient 14 through the cannula 20, when the speaking valve 26 is disposed on the connector 24, the cuff 48 should be deflated to enable the exhalation air to pass through the trachea 12 and larynx 32 and exit via the mouth 36.

As shown, the valve 26 aligns with the connector 24 such that a hollow body 50 of the valve 26 is concentric with the connector 24. The hollow body 50 may resemble a cylindrical annulus, and its distal end may couple with the connector 24 by an interference fit, snap fit, or other similar method. A cap 52 may be fitted over the proximal end of the hollow body 50 of the valve 26. The cap 52 may include alternating structural ribs 54 and openings 56. The ribs 54 may provide support for the structure of the cap 52 and/or hollow body 50, while the openings 56 may provide access to air for inhalation. Further, the ribs 54 may retain a valve member 58, which may be contained within the cap 52. The valve member 58 may be the component of the valve 26 that allows air to flow into the cannula 20 during inhalation and prevents air from exiting via the cannula 20 during exhalation. For example, the valve member 58 may be a membrane (e.g., diaphragm) that provides one-way deflection. However, in other embodiments, that valve member 58 may be a ball, disk, cone, wafer, or other suitable shape.

In certain embodiments, the body 50 and/or the valve member 58 may be formed of a moisture sensing material, particularly a material that undergoes a color change when subjected to moisture. The color change may be induced when the material reaches a specified saturation level. To provide the moisture induced color change, the body 50 and/or the valve member 58 may be constructed from a material that undergoes a chemical change, e.g., the color change, when exposed to moisture. Such materials may include temperature responsive polymers, pH sensitive polymers, halochromic materials, chromogenic materials, hydrogels, thermochromic materials, lichens based materials, other materials that may exhibit a color change due to exposure to moisture, or a combination thereof

The color change may provide a visual indication that moisture is accumulating within the valve 26, thereby indicating that the valve 26 needs to be cleaned or changed. In some embodiments, the listed materials may exhibit a gradual color change, with the degree of the color change indicating an approximate saturation level of the body 50 or the valve member 58. However, in other embodiments, the color change may only occur once the body 50 or the valve member 58 is completely saturated. In some embodiments, once saturated, the body 50 or the valve member 58 may be removed, discarded, and replaced with a new body 50 or valve member 58. This system may minimize the valve cleaning process, saving time and reducing the workload of caretakers. In alternative embodiments, the body 50 or the valve member 58 may simply be cleaned (e.g., sanitized, dried, etc.) and replaced within the valve 26. In this way, the components may be reused, reducing waste and cost associated with the valve 26. The valve 26 may be accompanied a guide indicating the range of the color change and which point in the color change signifies the valve 26 needs cleaning or replacement.

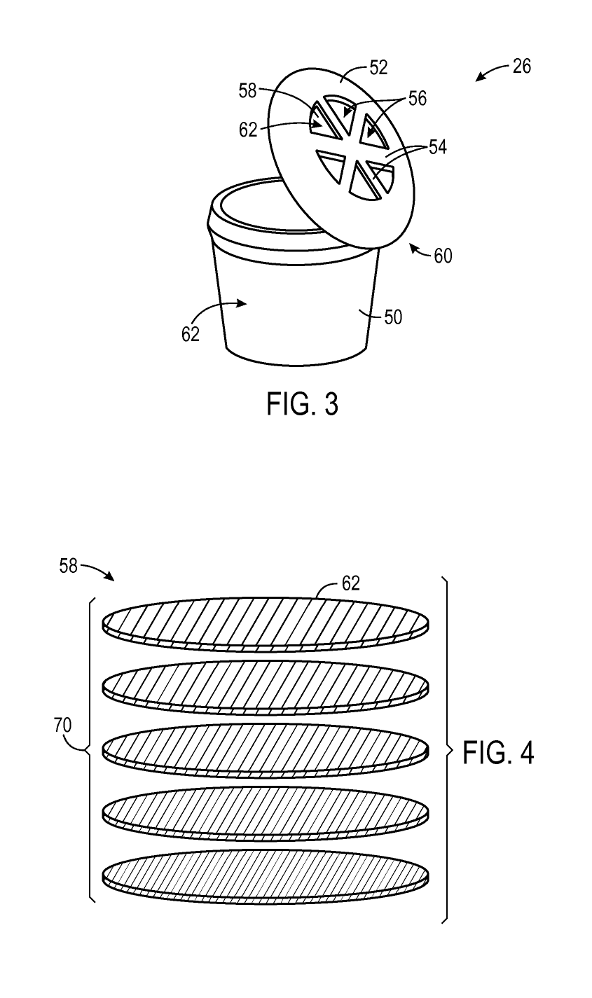

FIG. 3 provides a perspective view of an embodiment of the valve 26. In the depicted embodiment, the valve 26 includes a hinge 60 to couple the hollow body 50 with the cap 52. As shown, the valve member 58 may remain intact within the cap 52, such that it does not need to be removed separately. Thus, the hinge 60 may prevent loss of the cap 52 and the valve member 58 and may further enable the cap 52 to be spring loaded. The hinge 60 in conjunction with the spring loaded cap 52 may enable the valve 26 to be opened using minimal force, thereby preventing patient 14 discomfort when handling the valve 26. Further, when used with the cap 52 containing the valve member 58, the hinge 60 may provide access to the inside of the body 50 while the valve 26 remains coupled to the tracheostomy system 10 in the patient 14. In this manner, the hollow portion of the valve 26 may be quickly and easily accessed for cleaning As such, the hinge 60 may be particularly suitable for embodiments of the valve 26 including the valve member 58 as the color changing component.

In some embodiments of the valve 26, the hollow body 50 may be constructed from a hydrosensitive material 62 (e.g., a material that exhibits a color change when exposed to moisture). The entire body 50 may include the hydrosensitive material 62, or the hydrosensitive material 62 may only form a portion of the body 50 (e.g., an outer layer, an indicator ring, a pattern, etc.). Once the body 50 exhibits the color change, it may be optically viewed by the caretaker or patient 14. Due to the large area exhibiting the color change (e.g., the outside surface of the body 50), the color change may be easily perceived, resulting in prompt cleaning of the saturated valve 26. The saturated body 50 may be cleaned or discarded and replaced with a new body 50. The body 50 may be cleaned and/or replaced between approximately 1-10, 2-8, or 3-6 times per week. Within the valve 26, the body 50, the valve member 58, another component, or a combination thereof may include the hydrosensitive material 62.

As previously mentioned, in some embodiments, the valve member 58 may be formed of the material that exhibits a color change when exposed to moisture. In other words, the valve member 58 may incorporate the hydrosensitive material 62 in place of or in addition to the body 50. The valve member 58 may be constructed partially or entirely from the hydrosensitive material 62. For example, the valve member 58 may include a pattern formed from the hydrosensitive material 62 (e.g., woven, layered, geometrical, etc.). Once the valve member 58 exhibits the color change, it may be visually sensed by the caretaker or the patient 14 via the openings 56 in the cap 52. In this manner, the caretaker or patient 14 may monitor the moisture level of the valve 26 without physically handling the valve 26, minimizing contact with the tracheostomy system 10. Thus, the valve member 58 incorporating the hydrosensitive material 62 may provide the ability to visually inspect the valve 26 without touching it, thereby reducing the possibility of contamination or discomfort of the patient 14. As will be appreciated, the valve member 58 incorporating the hydrosensitive material 62 may act as a passive moisture indication system, which does not require power or prolonged interaction with the patient 14 or caretaker. The use of passive features may provide a simple and cost effective monitoring method, particularly as compared with electronic sensors.

As the valve member 58 becomes saturated (e.g., exhibits a specified level of color change), it may be removed from the valve 26 by opening the cap 52 via the hinge 60. The saturated valve member 58 may be discarded and replaced with a new (e.g., dry and clean) valve member 58, improving the cleanliness of the valve 26. However, in other embodiments, the saturated valve member 58 may be sanitized, dried, or otherwise cleaned, and replaced within the cap 52 of the valve 26, reducing the amount of waste associated with the valve 26. The valve member 58 may be cleaned and/or replaced between approximately 1-10, 2-8, or 3-6 times per week.

In embodiments having the valve member 58 as a color changing component, the valve member 58 may include multiple layers, as shown in FIG. 4. Particularly, in the depicted embodiment, that valve member 58 is a disk incorporating multiple membranes 70 in a layered arrangement. The membranes 70 may each include the hydrosensitive material 62. Further, each membrane 70 may include the same or different hydrosensitive materials 62. In this way, each layer of membrane 70 may provide a different color change scheme, which may be used to indicate progressing saturation levels within the valve 26. For example, as the last membrane 70 becomes saturated, it may undergo a color change to become red, signaling that the valve 26 is fully saturated and needs to be cleaned promptly.

In other embodiments, the layered structure may enable each membrane 70 to be removed as it becomes saturated and exhibits the color change due to the presence of the hydrosensitive material 62. In this manner, the time between cleanings for the valve 26 may be extended as the number of membranes 70 placed within the valve 26 increases.

For example, as each layer of membrane 70 exhibits a color change, it may be promptly removed to expose a different membrane 70, and so on until the valve member 58 is depleted and should be replaced. This method may result in consistently clean valves 26 since the moisture laden membranes 70 are removed upon saturation, thereby restoring clean and dry conditions within the valve 26. The embodiment of the valve member 58 in FIG. 4 includes five layers of membranes 70; however, any number between approximately 1 and 20 membranes 70 may be used in the valve 26.

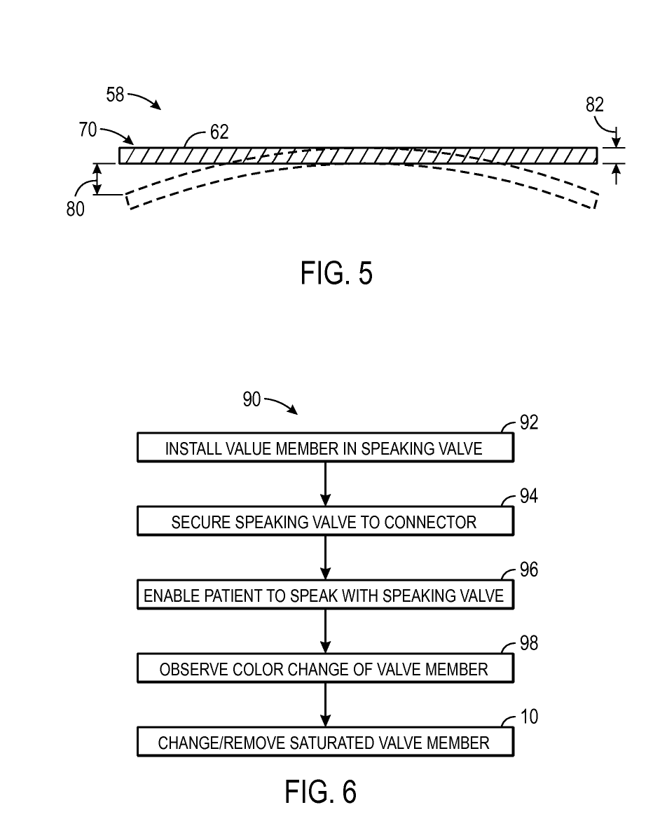

FIG. 5 provides a detailed view of a single membrane 70 made of the hydrosensitive material 62. As previously mentioned, the valve member 58 may be the component of the valve 26 that allows air to flow into the cannula 20 during inhalation and prevents air from exiting via the cannula 20 during exhalation. In certain embodiments, the valve member 58 formed from the membranes 70 may achieve such one-way flow by enabling one-way deflection 80. For example, the membranes 70 may be structured such that they are more susceptible to force applied in the direction of the inhalation air than in the direction of the exhalation air. In other words, the force applied to the membrane 70 by the inhalation air may cause the deflection 82 of the membrane, enabling inhalation air to travel through the cannula 20 to the lungs 16. However, the force applied to the membrane 70 by the exhalation air may not deflect the membrane 70, thereby forcing the air to exit the patient 14 through the larynx 32 and mouth 36 (e.g., enabling phonation). To provide adequate deflection 80 and enable the layered arrangement of the valve member 58 within the cap 52, the membranes 70 may have a limited thickness 82. For example, the thickness 82 of the membranes 70 may be between approximately 10 and 100 microns.

One embodiment of a method of use 90 for the speaking valve 26 with the tracheostomy system 10 is outlined in flow chart format in FIG. 6. In the presented embodiment, the valve member 58 (e.g., containing the hydrosensitive material 62) may be placed in the cap 52 of the speaking valve 26 (block 92). The speaking valve 26 may be secured to the connector 24 of the tracheostomy system 10 before or after the valve member 58 is placed in the cap 52 (block 94), particularly if the cap 52 includes the hinge 60. While the speaking valve 26 is secured to the tracheostomy system 10, the valve 26 prevents air from exiting the patient 14 via the cannula 20, thereby enabling the patient 14 to speak by forcing the air to travel through the vocal folds of the larynx 32 (block 96). As the patient 14 uses the valve 26 and the valve 26 becomes exposed to moisture in the breathing air and the airway of the patient 14, moisture may begin to saturate the valve member 58. When the saturation level of the valve member 58 (e.g., formed of the hydrosensitive material 62) exceeds a threshold value, the hydrosensitive material 62 may undergo a color change, which may be observed by the patient 14 or a caretaker (block 98). Upon observing the color change exhibited by the hydrosensitive material 62 in response to moisture build-up, the patient 14 or caretaker may be prompted to remove the saturated valve member 58 (e.g., to clean, discard, dry, etc.) (block 100). This method 90 may be further applied to embodiments wherein the hollow body 50 or any other component of the valve 26 is constructed from the hydrosensitive material 62. Further, additional steps may be included as part of the method 90, and the steps may be performed in a different sequence than presented.

Components of the tracheostomy system 10 and the speaking valve 26 may be manufactured according to suitable techniques. For example, the cannula 20, flange 22, connector 24, inflatable cuff 48, body 50, and the cap 52 may be molded, overmolded, two shot molded, blow molded, injection molded, or otherwise formed into the desired shape. Further, the listed components may be manufactured of materials such as a polyethylene (e.g., low density polyethylene), polypropylene, PTFE, expandable PTFE, polyvinyl chloride (PVC), a PEBAX silicone, a polyurethane, thermoplastic elastomers, a polycarbonate plastic, a silicon, or an acrylonitrile butadiene styrene (ABS). The membranes 70 formed of the hydrosensitive materials 62 may be punched, stamped, scored, cut, or otherwise made from a larger source (e.g., sheet, page, roll, etc.) of the hydrosensitive material 62.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Indeed, the disclosed embodiments may not only be applied to measurements of cuff pressure, but these techniques may also be utilized for the measurement and/or analysis of the tracheal pressure based on measurements of cuff pressure. Rather, the various embodiments may cover all modifications, equivalents, and alternatives falling within the spirit and scope of the disclosure as defined by the following appended claims.

What is claimed is:

1. A tracheostomy speaking valve, comprising:
a hollow body configured to be mounted to a tracheostomy tube connector;
a cap fitted over an end of the hollow body, the cap having ribs and openings between the ribs; and
a valve member disposed in the cap and occluding the openings to allow air to enter the tracheostomy tube during inhale cycles but to be forced through the larynx during exhale cycles;
wherein at least one of the hollow body, the cap and the valve member are made of a hydrosensitive material that undergoes a color change over time with exposure to moisture.

2. The valve of claim 1, wherein the valve member comprises multiple membranes arranged generally parallel to one another.

3. The valve of claim 2, wherein the valve membranes are configured to be removed successively as the membranes become moisture saturated.

4. The valve of claim 1, wherein the cap is hinged to the hollow body to permit opening of the cap and removal and/or replacement of the valve member.

5. The valve of claim 1, wherein the color change is irreversible.

6. The valve of claim 1, wherein the hydrosensitive material comprises a temperature responsive polymer, a pH sensitive polymer, a halochromic material, a chromogenic material, a hydrogel, a thermochromic material, a lichens based material, or any combination thereof.

7. The valve of claim 1, wherein the valve member has a thickness of from approximately 10 microns to approximately 100 microns.

8. The valve of claim 1, wherein the hollow body is configured to fit a 15 mm connector.

9. A tracheostomy system, comprising:
a tracheostomy tube assembly comprising a cannula configured to be disposed in the patient trachea, a flange configured to fit against the neck, and a connector extending from the flange; and
a speaking valve fitted to the connector and comprising a hydrosensitive material that undergoes a color change over time with exposure to moisture.

10. The system of claim 9, wherein the speaking valve comprises hollow body, a cap fitted over an end of the hollow body, the cap having ribs and openings between the ribs, and a valve member disposed in the cap and occluding the openings to allow air to enter the tracheostomy tube during inhale cycles but to be forced through the larynx during exhale cycles.

11. The system of claim 10, wherein the hollow body is at least partially made of the hydrosensitive material.

12. The system of claim 10, wherein the cap is at least partially made of the hydrosensitive material.

13. The system of claim 10, wherein the valve member is at least partially made of the hydrosensitive material.

14. The system of claim 9, wherein the speaking valve is removably secured to the connector.

15. The system of claim 9, wherein the tracheostomy tube assembly does not comprise an inflatable cuff.

16. A method comprising:
installing a valve member in a tracheostomy speaking valve, the valve member being at least partially made of a hydrosensitive material that undergoes a color change over time with exposure to moisture, the speaking valve being configured to be secured to a connector of a tracheostomy tube assembly; and
providing at least one replacement valve member that can replace the installed valve member as the installed valve member becomes saturated as indicated by the color change.

17. The method of claim 16, wherein the replacement valve member is made of the same material as the installed valve member.

18. The method of claim 16, wherein the color change is irreversible.

19. The method of claim 16, wherein the hydrosensitive material comprises a temperature responsive polymer, a pH sensitive polymer, a halochromic material, a chromogenic material, a hydrogel, a thermochromic material, a lichens based material, or any combination thereof

20. The method of claim 16, wherein the installed and the replacement valve members have a thickness of from approximately 10 microns to approximately 100 microns.
